(19) United States
(12) Patent Application Publication
(10) Pub. No.: US 2013/0164236 A1
(43) Pub. Date: Jun. 27, 2013

(54) ANTIPERSPIRANT COMPOSITIONS INCLUDING SILICA SORBED WITH DETERGENT AND METHODS OF MAKING SAME

(71) Applicants: Travis T. Yarlagadda, Phoenix, AZ (US); Tasha Desai, Phoenix, AZ (US); Chris Proulx, St. Piscataway, NJ (US); Gerald Decker, Aurora, IL (US); Matthew Romaine, Franklin Park, NJ (US); Saiid Mohammed, Somerset, NJ (US)

(72) Inventors: Travis T. Yarlagadda, Phoenix, AZ (US); Tasha Desai, Phoenix, AZ (US); Chris Proulx, St. Piscataway, NJ (US); Gerald Decker, Aurora, IL (US); Matthew Romaine, Franklin Park, NJ (US); Saiid Mohammed, Somerset, NJ (US)

(21) Appl. No.: 13/334,443

(22) Filed: Dec. 22, 2011

(51) Int. Cl. A61K 8/25; A61Q 15/00

(52) U.S. Cl. 424/65

(57) ABSTRACT

Antiperspirant compositions, products, and methods for making antiperspirant compositions and products are provided herein. In one embodiment, an antiperspirant composition includes an active antiperspirant compound. The antiperspirant composition also includes silica particles configured to at least partially dissolve in an alkaline environment. Further, an active detergent agent is sorbed by the silica particles and is configured to be released from the silica particles upon partial dissolution of the silica particles.

### TECHNICAL FIELD

The present invention relates generally to antiperspirant compositions and methods of making antiperspirant compositions, and more particularly relates to antiperspirant compositions that include silica sorbed with detergent to prevent or minimize fabric stains such as on garments worn by antiperspirant users.

### BACKGROUND

Antiperspirant and deodorant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, sticks, and soft solids, etc.

Fabric staining on garments worn by antiperspirant users, particular in the underarm area, has long been a concern with antiperspirant use. There are various factors that are believed to cause fabric staining by antiperspirant use. First, the acidic nature of typical active antiperspirant compounds in combination with perspiration may cause a fabric yellowing reaction to occur over time due to repeated and prolonged exposure. A second factor may be the presence of iron in the antiperspirant composition, such as in the active antiperspirant compound, clay, and/or fragrance, which can transfer to the garment and oxidize. Another factor is the presence of iron, calcium, and/or other inorganic metals found in the water used to wash a garment previously worn by the antiperspirant user. These inorganic metals can inhibit complete removal of the antiperspirant ingredients, resulting in a buildup of antiperspirant on the garment after multiple wearings and washings, and/or the inorganic metals can precipitate onto the garment to cause fabric staining.

Heretofore, efforts to address fabric staining typically have included incorporating less active antiperspirant compounds into the antiperspirant composition. However, many of these reduced active antiperspirant compounds lack antiperspirant efficacy relative to higher concentration active antiperspirant compounds. Also, fabric staining caused by factors other than the active antiperspirant compound, e.g., presence of iron in the antiperspirant composition, inorganic metals present in the wash water, antiperspirant buildup on the garment, and/or the like, are not addressed by simply using an antiperspirant composition with a less acidic active antiperspirant compound(s).

Accordingly, it is desirable to provide antiperspirant products that exhibit strong antiperspirant efficacy and that address fabric staining of garments worn by antiperspirant users. Also, it is desirable to provide antiperspirant compositions that incorporate silica particles sorbed with detergent and configured to release the detergent in a washing environment. Furthermore, other desirable features and characteristics of the present invention will become apparent from the subsequent detailed description of the invention and the appended claims, taken in conjunction with the accompanying drawings and this background.

### BRIEF SUMMARY

Antiperspirant compositions and products, and methods for making antiperspirant compositions and products are provided herein. In an exemplary embodiment, an antiperspirant composition comprises an active antiperspirant compound and silica particles configured to at least partially dissolve in an alkaline environment. An active detergent agent is sorbed by the silica particles and is configured to be released from the silica particles upon partial dissolution of the silica particles.

In accordance with another exemplary embodiment, an antiperspirant product is provided. The antiperspirant product comprises a container and an antiperspirant composition housed within the container. The antiperspirant composition includes an active antiperspirant compound and silica particles configured to at least partially dissolve in an alkaline environment. An active detergent agent is sorbed by the silica particles and configured to be released from the silica particles upon partial dissolution of the silica particles.

In accordance with another exemplary embodiment, a method for making an antiperspirant composition is provided. The method comprises providing silica particles and sorbing an active detergent agent with the silica particles. In the method, the silica particles are mixed with antiperspirant ingredients including an active antiperspirant compound to form an antiperspirant composition.

### BRIEF DESCRIPTION OF THE DRAWING

The present invention will hereinafter be described in conjunction with the following drawing figure, wherein:

FIG. 1 is a perspective view of an antiperspirant product in accordance with an exemplary embodiment.

### DETAILED DESCRIPTION

The following Detailed Description is merely exemplary in nature and is not intended to limit the invention or the application and uses of the invention. Furthermore, there is no intention to be bound by any theory presented in the preceding background or the following detailed description.

The various embodiments contemplated herein relate to antiperspirant compositions that exhibit strong antiperspirant efficacy and remove or inhibit fabric stains, antiperspirant products including such antiperspirant compositions, and methods for making such antiperspirant compositions. Unlike the prior art, the exemplary embodiments herein include an antiperspirant composition with an active antiperspirant compound and with an active detergent agent that is sorbed by silica particles. As used herein, “sorbed” means held, as by absorption into or adsorption onto, by another substance. In other words, the active detergent agent may be absorbed into and/or adsorbed onto the silica particles.

The active antiperspirant compound is effective to prevent the secretion of perspiration and/or accompanying odors. The active detergent agent is effective to remove or inhibit fabric stains, such as on a garment worn by an antiperspirant user. The active detergent agent is sorbed onto silica particles to delay its release until the silica dissolves, such as in an alkaline environment like that in washing machine. Upon release, the active detergent agent is exposed and can remove or inhibit a fabric stain on the garment that may have been caused by antiperspirant use. Further, the active detergent agent is located on the garment where the antiperspirant may fond a stain. Therefore, the release of the active detergent agent is targeted to the location of antiperspirant-caused stain formation.

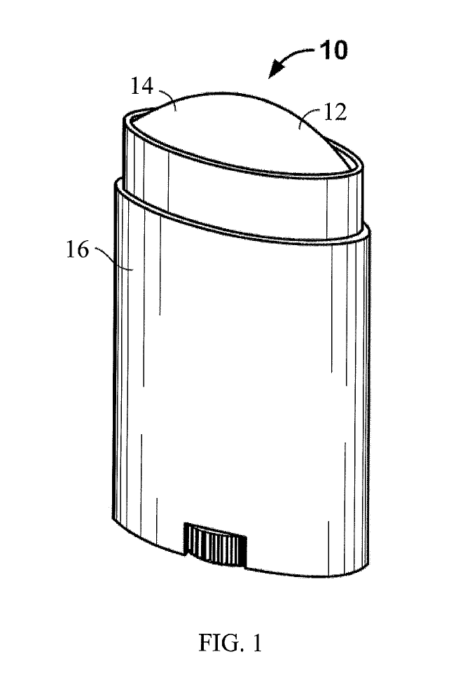

Referring to FIG. 1, an antiperspirant product 10 in accordance with an exemplary embodiment is provided. The antiperspirant product 10 comprises an antiperspirant composition 12, such as a solid wax antiperspirant formulation. As illustrated, the antiperspirant composition 12 has an application surface 14 that is substantially dome-shaped and that is configured to be applied to skin, such as, for example, an underarm. The antiperspirant product 10 may also comprise a container or dispenser 16 for dispensing the antiperspirant composition 12 to the skin.

The antiperspirant composition 12 contains at least one active ingredient (i.e. active antiperspirant compound), typically metal salts, that are thought to reduce perspiration by diffusing through the sweat ducts of eccrine glands and apocrine glands and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, such as inorganic and organic salts of aluminum, zirconium, and zinc, as well as mixtures thereof. Some examples are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Some zirconium salt examples are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof.

The active antiperspirant compound is preferably in a perspiration-reducing effective amount. In one embodiment, the antiperspirant composition 12 comprises an active antiperspirant compound present in the amount of from about 5 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycine.

The antiperspirant composition 12 contains an active detergent agent that is effective to remove or inhibit fabric stains. The active detergent agent is sorbed by silica particles. In an exemplary embodiment, the silica particle sorbed with the active detergent agent are present in an amount of from about 0.1 to about 8 wt. % of the antiperspirant composition 12. In an exemplary embodiment, the active detergent agent forms about 50 wt. % to about 90 wt. % of the silica particles sorbed with detergent.

In an exemplary embodiment, the silica particles are hydrophobic. “Hydrophobic” silica particles, as the term is used herein, encompasses silica particles having varying levels or degrees of hydrophobicity. The degree of hydrophobicity imparted to the silica particles will vary depending upon the type and amount of treating agent used.

Preferably, hydrophobic silica particles are formed from treated silica particles, such as by fuming or co-fuming with silanes or siloxanes. The silica particles may be produced utilizing techniques known to those skilled in the art. The production of a fumed metal oxide is a well-documented process which involves the hydrolysis of suitable feed stock vapor (such as silicon tetrachloride) in a flame of hydrogen and oxygen. Molten particles of roughly spherical shape are formed, and the particle diameters may be varied through control of process parameters. These molten spheres, referred to as primary particles, fuse with one another by undergoing collisions at their contact points to form branched, three dimensional chain-like aggregates. The formation of the aggregates is considered to be irreversible as a result of the fusion between the primary particles. During cooling and collecting, the aggregates undergo further collisions that may result in some mechanical entanglements to form agglomerates. These agglomerates are thought to be loosely held together by van der Waals forces and can be reversed, i.e. de-agglomerated, by proper dispersion in a suitable media. Mixed or co-fumed silica particles may also be produced utilizing conventional techniques known to those skilled in the art. The silica particles described herein may include other oxides such as those of aluminum, titanium, zirconium, iron, niobium, vanadium, tungsten, tin, or germanium. Such aggregates may be formed by introducing appropriate feed stocks (e.g. chloride compounds) into a flame in conjunction with an appropriate fumed silica feed stock. A non-limiting example of fumed silica particles includes AEROSIL® fumed silica available from Evonik Corporation.

Treatment of silicon dioxide particles refers to the chemical modification of the surface silanol functionality. As covered extensively in literature, there are many mechanisms that allow for surface modification via various chemical reaction routes and processes. When discussing treated silica particles it is important to understand not only the physical properties but to also understand the chemical structure at the surface. This can be seen in effect with AEROSIL® R 816 (hexadecyl-silane treated) which creates a unique balance between hydrophilic (silanol functionality) and hydrophobic (treated chemical functionality) properties.

Chemical selectivity with actives is also influenced by the degree of chemical modification at the particle's surface. One example of this can be seen between AEROSIL® R 812 and 812S (Hexamethyldisilazane). Both products have identical treatments however the degree of silanol group substitution is less with AEROSIL® R 812. This can shift how particles interact within a given matrix. These chemical differences in conjunction with the previously described physicochemical properties form the foundation for specific interactions between inorganic and organic components within applications.

In exemplary embodiments, the treated silica particles may have a BET surface area (ASTM D6556-07) of about 35 m²/g to about 700 m²/g, for example, greater than about 60 m²/g, greater than about 80 m²/g, greater than about 130 m²/g, or greater than about 170 m²/g; less than about 400 m²/g, less than about 290 m²/g, less than about 250 m²/g; or about 200 m²/g.

In an exemplary embodiment, the active detergent agent comprises a surfactant, an acidic detergent component, an alkaline builder, a water conditioner, an antioxidant, a soil release polymer, an oxidizing agent, an enzyme, a corrosion inhibitor, glycol ether, and/or butylcellosolve. The term “surfactant” as used herein in reference to the active detergent agent refers to a detergent agent ingredient(s) that lowers the surface tension of water, e.g., perspiration or wash water, so that the water is more likely to interact with soil materials, e.g., material(s) causing or forming a fabric stain, to remove or inhibit a fabric stain. The term “acidic detergent component” refers to a detergent agent ingredient(s) that lowers the pH of water (e.g. perspiration or wash water) to a pH range that facilitates preventing, removing or minimizing a fabric stain. The term “alkaline builder” as used herein refers to a detergent agent ingredient(s) that increases, buffers, and/or stabilizes the pH of water (e.g. perspiration or wash water) to a pH range that facilitates preventing, removing or minimizing a fabric stain. The term “water conditioner” as used herein refers to a detergent agent ingredient(s), such as a sequestering agent and/or a chelating agent, that neutralizes, combines with, and/or removes iron, calcium, and/or other inorganic metals in water (e.g. perspiration or wash water), and/or helps remove antiperspirant buildup to remove or inhibit a fabric stain. The term “antioxidant” as used herein refers to a detergent agent ingredient(s) that reduces or prevents oxidation of iron and/or other inorganic metals in water and/or antiperspirant to remove or inhibit a fabric stain. The term “soil release polymer” as used herein refers to a polymeric detergent agent ingredient(s) that protects the fibers of a fabric by reducing the affinity of soil materials to cling to a fabric surface to remove or inhibit a fabric stain. The term “oxidizing agent” as used herein refers to a detergent agent ingredient(s) that can oxidize and/or react with soil materials to remove or inhibit a fabric stain. The term “enzyme” as used herein refers a microorganism detergent agent ingredient(s) that facilitates preventing, removing or minimizing a fabric stain. The term “corrosion inhibitor” as used herein refers to a detergent agent ingredient(s) that prevents corrosion or oxidation of inorganic metals in water (e.g. perspiration or wash water) to remove or inhibit a fabric stain.

Some examples of surfactants suitable as detergent agent ingredients include surfactants having a head group that is anionic, cationic, amphoteric, or nonionic, and a tail group. Examples of ionic head groups include head groups having a negative charge and comprising sulfates, sulfonates, phosphates, carboxylates, and/or the like. Examples of cationic head groups include head groups having a positive charge and comprising amines, quaternary ammonium cation, and/or the like. Examples of amphoteric head groups include head groups having both a positive charge and a negative charge, and comprising sulfonates, carboxylates, phosphates, and/or the like. Examples of nonionic head groups include head groups having no charge and comprising fatty alcohols and/or the like. The term “fatty” as used herein is intended to include hydrocarbon chains of about 8 to 30 carbon atoms, such as about 12 to 18 carbon atoms. Examples of tail groups include tail groups comprising hydrocarbon chain(s), alkyl ether chain(s) such as an ethoxylated or propoxylated chains, fluorocarbon chain(s), siloxane chain(s), and/or the like. In an exemplary embodiment, the detergent agent comprises fatty alcohol ethoxylate as a surfactant.

Some examples of acidic detergent components include phosphoric acid, nitric acid, sulfamic acid, sodium acid sulfate, hydrochloric acid, hydroxyacetic acid, citric acid, gluconic acid, and/or the like. Some examples of alkaline builders include sodium hydroxide, potassium hydroxide, tri-sodium phosphate, alkaline builder salts, and/or the like. Examples of alkaline builder salts include sodium, potassium, or ammonium salts of phosphates, silicates, or caronates. Some examples of water conditioners include sequestering agents and/or chelating agents. Examples of sequestering agents include sodium tripolyphosphate, tetra-potassium pyrophosphate, organo-phosphates, polyelectrolytes, and/or the like. Examples of chelating agents include sodium gluconate, ethylene diamine tetracidic acid, and/or the like. Some examples of oxidizing agents include sodium gluconate, ethylene diamine tetracidic acid, and/or the like. Some examples of enzymes include protease, lipase, amylase, mannanase, and/or the like.

It is herein disclosed that sorbing the active detergent agent with the silica particles allows the active detergent agent to be held for release from the silica particles upon introduction to a selected environment to remove or inhibit formation of a fabric stain. For example, the silica particles are configured to dissolve in an alkaline environment having, for example, a pH of at least 8, of at least 9, or of at least 10. Typical laundry detergent provides such an alkaline environment during washing. Therefore, the active detergent agent may be held by the silica particles until they at least partially dissolve in the mixture of water and laundry detergent in a washing machine. Upon partial dissolution of the silica particles, the active detergent agent is released from the antiperspirant on the garment, and may remove or inhibit formation of a stain on the garment.

The antiperspirant composition 12 may further comprise an anhydrous, hydrophobic vehicle, which includes a volatile silicone and/or a high melting component. In an exemplary embodiment, the active antiperspirant compound is suspended in the anhydrous, hydrophobic vehicle.

For use as an antiperspirant stick, the high melting components may include any suitable material suitable that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, such as from about 2 to about 15 wt. %, of the antiperspirant composition 12. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant composition 12 contemplated herein also may comprise additives, such as those used in conventional antiperspirants. For example, in addition to antiperspirant efficacy, the antiperspirant composition 12 may comprise additives that cause the antiperspirant composition 12 to exhibit long-lasting fragrance, odor protection, bacteria control, and/or another desired purpose and/or function. These additives include, but are not limited to, fragrances, including encapsulated fragrances, dyes, pigments, preservatives, antioxidants, moisturizers, and the like. These optional ingredients can be included in an amount of from about 0 to about 20 wt. % of the antiperspirant composition 12.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as particulate polyolefins, talcum materials, colorants and preservatives may also be included as desired. For example, the antiperspirant composition 12 may include up to about 5% fragrance or about 2% colorant by weight.

As noted above, in addition to an active antiperspirant compound, the antiperspirant composition 12 may comprise a component or components that cause it to exhibit or impart a desired function or purpose in addition to antiperspirant efficacy. For example, the antiperspirant composition 12 may comprise deodorant active ingredients. A suitable deodorant active ingredient is any agent that inhibits, suppresses, masks or neutralizes malodor. These may include (1) antimicrobial or bactericidal agents that kill the bacteria responsible for malodor production, (2) agents that inhibit or suppress or interfere with the bacterial enzymatic pathway that produces malodor, and (3) agents that mask or absorb or neutralize malodor. “Fragrances” as used herein are not considered deodorant active ingredients. Examples of deodorant actives ingredients include triclosan, triclocarban, usnic acid salts, zinc phenolsulfonate, b-chloro-D-alanine, D-cycloserine, animooxyacetic acid, cyclodextrine, and sodium bicarbonate. Alternatively, or in addition, the antiperspirant composition 12 may comprise fragrances, for example, in an amount that imparts a long-lasting fragrance to the antiperspirant composition 12.

In accordance with exemplary embodiments, a method for making the antiperspirant composition includes providing silica particles and sorbing an active detergent agent with the silica particles. The method further includes mixing the silica particles with antiperspirant ingredients including an active antiperspirant compound to form the antiperspirant composition. Other suitable methods for forming the antiperspirant composition known to those skilled in the art may also be used.

The following are examples of an antiperspirant product in accordance with exemplary embodiments, including an invisible solid product, an aerosol product, a roll-on product, and a gel product. The examples are provided for illustration purposes only and are not meant to limit the various embodiments of the antiperspirant product in any way. All materials are set forth in weight percent.

### EXAMPLE 1

### Antiperspirant Product—Invisible Solid Formulation

| Ingredient | Wt. % |
|---|---|
| Aluminum Zirconium | 5 to 25 |
| Pentachlorohydrex | |
| Cyclopentasiloxane | 32 to 46 |
| Stearyl Alcohol | 15 to 24 |
| PPG-14 Butyl Ether | 7 to 15 |
| Hydrogenated Castor Oil | 1 to 8 |
| Myristal Myristate | 1 to 8 |
| Silica | 0.1 to 6 |
| Silica Dimethyl Silylate | 0.1 to 6 |
| Parfum | 0.1 to 6 |
| Silica sorbed with Detergent | 0.1 to 8 |
| Total | 100.0 |

### EXAMPLE 2

### Antiperspirant Product—Aerosol Formulation

| Ingredient | Wt. % |
|---|---|
| Butane | 30 to 45 |
| Cyclomethicone | 20 to 35 |
| Hydrofluorocarbon 152 | 10 to 25 |
| Aluminum Chlorohydrate | 6 to 25 |
| Talc | 0.5 to 4 |
| Silica Dimethyl Silylate | 0.5 to 4 |
| Fragrance | 0.5 to 2 |
| Silica | 0.1 to 1 |
| Silica sorbed with Detergent | 0.1 to 8 |
| Total | 100.0 |

### EXAMPLE 3

### Antiperspirant Product—Roll-On Formulation

| Ingredient | Wt. % |
|---|---|
| Aluminum Zirconium | 10 to 25 |
| Pentachlorohydrex - GLY | |
| Cyclomethicone | 40 to 80 |
| Disteardimonium Hectorite | 1 to 10 |
| Tocopheryl Acetate | 0.1 to 3 |
| Propylene Carbonate | 0.1 to 3 |
| Fragrance | 0.1 to 3 |
| Silica sorbed with Detergent | 0.1 to 8 |
| Total | 100.0 |

### EXAMPLE 4

### Antiperspirant Product—Gel Formulation

| Ingredient | Wt. % |
|---|---|
| Aluminum Zirconium | 10 to 25 |
| Octachlorohydrex GLY | |
| Water | 20 to 50 |
| Ethanol | 5 to 15 |
| Cyclomethicone | 1 to 12 |
| PEG/PPG-18/18 Dimethicone | 1 to 12 |
| Propylene Glycol | 1 to 12 |
| Fragrance | 0.1 to 3 |
| Silica sorbed with Detergent | 0.1 to 8 |
| Total | 100.0 |

The following is an example of an active detergent agent sorbed by silica particles in accordance with exemplary embodiments. The example is provided for illustration purposes only and is not meant to limit the various embodiments of the antiperspirant composition in any way. All materials are set forth in weight percent.

### EXAMPLE 5

### Silica Particles Sorbed with Active Detergent Agent

| Ingredient | Wt. % |
|---|---|
| Alkyl Polyglucoside | 1 to 8 |
| Fatty Alcohol Ethoxylate C12-14 | 0.01 to 2 |
| Water | 32 to 78 |
| Sodium Citrate | 0.0 to 6 |
| Phosphoric Acid | 0.5 to 8 |
| Citric Acid | 2 to 15 |
| Formic Acid | 0.1 to 6 |
| Sodium Hydroxide | 0.1 to 6 |
| Polydimethyl Siloxane | 0.0 to 1 |
| Zinc Ricinoleate | 0.05 to 6 |
| Silica Particles | 10 to 50 |
| Total | 100.0 |

Accordingly, antiperspirant compositions that exhibit strong antiperspirant efficacy and that are effective to remove or inhibit formation of fabric stains, antiperspirant products comprising such antiperspirant compositions, and methods for making such antiperspirant compositions have been described. Unlike the prior art, the exemplary embodiments taught herein form an antiperspirant composition that comprises an active antiperspirant compound plus an active detergent agent that is sorbed by silica particles. The active antiperspirant compound is effective to inhibit secretion of perspiration and/or accompanying odors. The active detergent agent is effective to remove or inhibit formation of fabric stains, such as, for example, on a garment worn by an antiperspirant user.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the processes without departing from the scope defined by the claims, which includes known equivalents and foreseeable equivalents at the time of filing this patent application.

What is claimed is:

1. An antiperspirant composition comprising:
an active antiperspirant compound;
silica particles configured to at least partially dissolve in an alkaline environment; and
an active detergent agent sorbed by the silica particles and configured to be released from the silica particles upon partial dissolution of the silica particles.

2. The antiperspirant composition of claim 1 wherein the silica particles are hydrophobic.

3. The antiperspirant of claim 2 wherein the silica particles are chemically treated with silanes or siloxanes.

4. The antiperspirant composition of claim 1 wherein the silica particles are configured to be at least partially dissolved in an alkaline environment having a pH of at least 8.

5. The antiperspirant composition of claim 1 wherein the silica particles are configured to be at least partially dissolved in an alkaline environment having a pH of at least 9.

6. The antiperspirant composition of claim 1 wherein the silica particles are configured to be at least partially dissolved in an alkaline environment having a pH of at least 10.

7. The antiperspirant composition of claim 1 wherein the silica particles and detergent agent comprise about 0.1 to about 8 wt. % of the antiperspirant composition.

8. The antiperspirant composition of claim 1 wherein the active detergent agent comprises a surfactant, an acidic detergent component, an alkaline builder, a water conditioner, an antioxidant, a soil release polymer, an oxidizing agent, an enzyme, a corrosion inhibitor, glycol ether, butylcellosolve, or combinations thereof.

9. The antiperspirant composition of claim 8 wherein the surfactant comprises sulfates, sulfonates, phosphates, carboxylates, amines, quaternary ammonium cation, fatty alcohols, or combinations thereof.

10. The antiperspirant composition of claim 8 wherein the acidic detergent component comprises phosphoric acid, nitric acid, sulfamic acid, sodium acid sulfate, hydrochloric acid, hydroxyacetic acid, citric acid, gluconic acid, or combinations thereof.

11. The antiperspirant composition of claim 8 wherein the alkaline builder comprises sodium hydroxide, potassium hydroxide, tri-sodium phosphate, alkaline builder salts, or combinations thereof, wherein the alkaline builder salts comprise sodium, potassium, or ammonium salts of phosphates, silicates, or caronates.

12. The antiperspirant composition of claim 8 wherein the water conditioner comprises a sequestering agent, a chelating agent, or combinations thereof, wherein the sequestering agent comprises sodium tripolyphosphate, tetra-potassium pyrophosphate, organo-phosphates, polyelectrolytes, or combinations thereof, and wherein the chelating agent comprises sodium gluconate, ethylene diamine tetracidic acid, or combinations thereof.

13. The antiperspirant composition of claim 8 wherein the oxidizing agent comprises hypochlorite, perborate, or combinations thereof.

14. The antiperspirant composition of claim 8 wherein the enzyme comprises protease, lipase, amylase, mannanase, or combinations thereof.

15. An antiperspirant product comprising:
a container; and
an antiperspirant composition housed within the container, wherein the antiperspirant composition includes:
an active antiperspirant compound;
silica particles configured to at least partially dissolve in an alkaline environment; and
an active detergent agent sorbed by the silica particles and configured to be released from the silica particles upon partial dissolution of the silica particles.

16. The antiperspirant product of claim 15 wherein the silica particles sorbed with the active detergent agent are present in an amount of from about 0.1 to about 8 wt. % of the antiperspirant composition.

17. The antiperspirant product of claim 15 wherein the silica particles are hydrophobic.

18. The antiperspirant product of claim 15 wherein the active detergent agent comprises a surfactant, an acidic detergent component, an alkaline builder, a water conditioner, an antioxidant, a soil release polymer, an oxidizing agent, an enzyme, a corrosion inhibitor, glycol ether, butylcellosolve, or combinations thereof.

19. The antiperspirant product of claim 15 wherein the silica particles are chemically treated with silanes or siloxanes.

20. A method for making an antiperspirant composition comprising:
providing silica particles;
sorbing an active detergent agent with the silica particles; and
mixing the silica particles with antiperspirant ingredients including an active antiperspirant compound to form the antiperspirant composition.
